(19) United States
(12) Patent Application Publication
(10) Pub. No.: US 2016/0137583 A1
(43) Pub. Date: May 19, 2016

(54) METHOD FOR PRODUCING ALPHA-HYDROXYCARBOXYLIC ACID ESTERS

(71) Applicants: Steffen KRILL, Mühltal (DE); Alexander MAY, Seeheim-Jugenheim (DE); Jörg BECKER, Karlsruhe (DE); Martin KÖSTNER, Darmstadt (DE); EVONIK RÖHM GMBH, Darmstadt (DE)

(72) Inventors: Steffen KRILL, Muehltal (DE); Alexander MAY, Seeheim-Jugenheim (DE); Joerg BECKER, Karlsruhe (DE); Martin KOESTNER, Darmstadt (DE)

(73) Assignee: EVONIK ROHM GMBH, Darmstadt (DE)

(21) Appl. No.: 14/900,403

(22) PCT Filed: Jul. 3, 2014
(86) PCT No.: PCT/EP2014/064194

§ 371 Date: Dec. 21, 2015

(30) Foreign Application Priority Data
Jul. 12, 2013 (DE) 10 2013 213 699.4

(51) Int. Cl. C07C 67/20

(52) U.S. Cl. CPC C07C 67/20

(57) ABSTRACT

The present invention relates to a process for the production of alpha-hydroxycarboxylic esters by means of alcoholysis of the corresponding alpha-hydroxycarboxamide under heterogeneous catalysis.

The present invention relates to a process for the production of alpha-hydroxycarboxylic esters by means of alcoholysis of the corresponding alpha-hydroxycarboxamide under heterogeneous catalysis.

Various processes for the alcoholysis of alpha-hydroxycarboxamides (aHCA) are known from the prior art. The literature, for example, describes both processes catalysed homogeneously with lanthanum or titanium compounds (Canadian Journal of Chemistry, 1994. 72(1): p. 142-145; Canadian Journal of Chemistry, 2004. 82(12): p. 1791-1805) and processes catalysed hetereogeneously over highly acidic ion exchangers or aluminium oxide (Bulletin of the Korean Chemical Society, 1997. 18(11): p. 1208-1210). A further process based on homogeneous catalysis is claimed in DE 102011081256. In addition to the dominant homogeneous mode of operation, EP 945423 also refers to examples with insoluble metal oxides such as bismuth oxides or cerium oxides and also bismuth metal. JP 08073406 and JP 06345692 are directed exclusively at a heterogeneous catalysis with metal oxides used in unsupported form or applied to SiO₂ supports. Antimony oxide, tellurium oxide, bismuth oxide and zirconium oxide are cited as particularly useful.

Whilst the homogeneous processes are associated with significant disadvantages such as
little or no tolerance with regard to the presence of water with the attendant unstable operation and frequent interruptions due to blockages and precipitations (e.g. lanthanum salts, inter alia, hydroxide, oxide and/or hydrates thereof) in the workup section,
resulting low plant availability and
high catalyst and disposal costs due to the necessary discharging of the homogeneous catalyst,
   problems precluding economic operation frequently occur in the heterogeneous processes during the recirculation of unreacted starting materials contaminated with by-products.

It is therefore an object of the present invention to provide a process optimized for high single path conversions based on the reactant aHCA, which is highly robust and therefore allows long operating lifetimes also in circulation mode. Further, the process should have a high tolerance to the presence of water and by-products introduced into the reaction from the previous stage with the reactant, coupled with the option of a circulation mode which allows the unreacted reactants and/or by-products from the alcoholysis to be fed back into the reaction without any pre-purification, and in particular allows the former to be converted into the desired product of value without efficiency losses.

This object is achieved by a continuous process for the production of alpha-hydroxycarboxylic esters by alcoholysis of the corresponding alpha-hydroxycarboxamide, characterized in that
a) Reactant streams comprising an alpha-hydroxycarboxamide and an alcohol are fed into a pressure reactor containing a heterogeneous catalyst,
b) this reaction mixture is co-reacted in the pressure reactor at a pressure in the range of 1-100 bar in the liquid phase,
c) the product mixture resulting from step b) comprising alpha-hydroxycarboxylic ester and also unreacted alpha-hydroxycarboxamide is discharged from the pressure reactor,
d) the product mixture resulting from c) is depleted in alcohol and ammonia and
e) the unreacted reactants and also by-products comprising ammonium salts of the corresponding alpha-hydroxycarboxylic acid, present in the product mixture from step d), are recycled to step a).
   The described disadvantages of the prior art are thereby partially or completely eliminated and an economic process with a high space-time yield is provided.

aHCAs that may be used for the purposes of the invention include carboxamides having at least one hydroxyl group in the alpha-position to the carboxamide group.

Carboxamides are common knowledge in the technical field. Typically, these are understood to mean compounds having groups of the formula —CONR′R″ in which R′ and R″ are each independently hydrogen or a group having 1-30 carbon atoms which in particular comprises 1-20, preferably 1-10 and in particular 1-5 carbon atoms. The carboxamide may comprise 1 to 4 or more groups of the formula —CONR′R″. These include in particular compounds of the formula R(—CONR′R″)n in which the R radical is a group having 1 to 30 carbon atoms, which in particular has 1 to 20, preferably 1 to 10, particularly 1 to 5 and more preferably 2 to 3 carbon atoms, R′ and R″ are as defined above and n is an integer in the range of 1 to 10, preferably 1 to 4 and more preferably 1 or 2.

The expression “group having 1 to 30 carbon atoms” denotes radicals of organic compounds having 1 to 30 carbon atoms. In addition to aromatic and heteroaromatic groups, it also includes aliphatic and heteroaliphatic groups, such as, for example, alkyl, cycloalkyl, alkoxy, cycloalkoxy, cycloalkylthio and alkenyl groups. The groups mentioned may be branched or unbranched.

According to the invention, aromatic groups denote radicals of mono- or polycyclic aromatic compounds having preferably 6 to 20, in particular 6 to 12, carbon atoms.

Heteroaromatic groups denote aryl radicals in which at least one CH group has been replaced by N and/or at least two adjacent CH groups have been replaced by S, NH or O.

Aromatic or heteroaromatic groups preferred in accordance with the invention derive from benzene, naphthalene, biphenyl, diphenyl ether, diphenylmethane, diphenyldimethylmethane, bisphenone, diphenyl sulphone, thiophene, furan, pyrrole, thiazole, oxazole, imidazole, isothiazole, isoxazole, pyrazole, 1,3,4-oxadiazole, 2,5-diphenyl-1,3,4-oxadiazole, 1,3,4-thiadiazole, 1,3,4-triazole, 2,5-diphenyl-1,3,4-triazole, 1,2,5-triphenyl-1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benzo[c]thiophene, benzo[c]furan, isoindole, benzoxazole, benzothiazole, benzimidazole, benzisoxazole, benzisothiazole, benzopyrazole, benzothiadiazole, benzotriazole, dibenzofuran, dibenzothiophene, carbazole, pyridine, bipyridine, pyrazine, pyrazole, pyrimidine, pyridazine, 1,3,5-triazine, 1,2,4-triazine, 1,2,4,5-triazine, tetrazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine or quinolizine, 4H-quinolizine, diphenyl ether, anthracene, benzopyrrole, benzooxathiadiazole, benzooxadiazole, benzopyridine, benzopyrazine, benzopyrazidine, benzopyrimidine, benzotriazine, indolizine, pyridopyridine, imidazopyrimidine, pyrazinopyrimidine, carbazole, acridine, phenazine, benzoquinoline, phenoxazine, phenothiazine, acridizine, benzopteridine, phenanthroline and phenanthrene, each of which may also optionally be substituted.

The preferred alkyl groups include the methyl, ethyl, propyl, isopropyl, 1-butyl, 2-butyl, 2-methylpropyl, tert-butyl, pentyl, 2-methylbutyl, 1,1-dimethylpropyl, hexyl, heptyl, octyl, 1,1,3,3-tetramethylbutyl, nonyl, 1-decyl, 2-decyl, undecyl, dodecyl, pentadecyl and the eicosyl group.

The preferred cycloalkyl groups include the cyclopropyl, cyclobutyl, cyclopentyl, cyclohexyl, cycloheptyl and the cyclooctyl group, each of which may optionally be substituted with branched or unbranched alkyl groups.

The preferred alkenyl groups include the vinyl, allyl, 2-methyl-2-propenyl, 2-butenyl, 2-pentenyl, 2-decenyl and the 2-eicosenyl group.

The preferred heteroaliphatic groups include the aforementioned preferred alkyl and cycloalkyl radicals in which at least one carbon unit has been replaced by O, S or an NR′ or NR¹R² group, and R¹ and R² are each independently an alkyl group having 1 to 6 carbon atoms, an alkoxy group having 1 to 6 carbon atoms or an aryl group.

In accordance with the invention, very particular preference is given to carboxamides having branched or unbranched alkyl or alkoxy groups having 1 to 20 carbon atoms, preferably 1 to 12, advantageously 1 to 6, in particular 1 to 4 carbon atoms, and cycloalkyl or cycloalkyloxy groups having 3 to 20 carbon atoms, preferably 5 to 6 carbon atoms. These may be substituted. Preferred substituents include halogens, in particular fluorine, chlorine, bromine, and also alkoxy or hydroxyl radicals.

The alpha-hydroxycarboxamides may be used individually or as a mixture of two or more different aHCA in the process according to the invention. Particularly preferred aHCA include alpha-hydroxyisobutyramide and/or alpha-hydroxyisopropionamide.

Furthermore, in a modification of the process according to the invention, it is of particular interest to use alpha-hydroxycarboxam ides obtainable by cyanohydrin synthesis from ketones or aldehydes and hydrogen cyanide. In a first step, the carbonyl compound, for example a ketone, in particular acetone, or an aldehyde, for example acetaldehyde, propanal or butanal, is reacted with hydrogen cyanide to form the respective cyanohydrin. Particular preference is given to reacting acetone and/or acetaldehyde in a conventional manner using a small amount of alkali or of an amine as catalyst. In a further step, the cyanohydrin thus obtained is reacted with water to give the aHCA.

This reaction is typically carried out in the presence of a catalyst. Useful catalysts for this purpose are in particular manganese oxide catalysts, as described, for example, in EP-A-0945429, EP-A-0561614, EP-A-0545697 and also EP 2268396. The manganese oxide may be used in the form of manganese dioxide, which is obtained by treating manganese sulphate with potassium permanganate under acidic conditions (cf. Biochem. J., 50, p. 43 (1951) and J. Chem. Soc., 1953, p. 2189, (1953)) or by electrolytic oxidation of manganese sulphate in aqueous solution. In general, the catalyst is often used in the form of powder or granules having a suitable particle size.

Alcohols that may be used in processes of the invention include all alcohols familiar to those skilled in the art and also precursor compounds of alcohols which, under the conditions of pressure and temperature indicated, are capable of reacting with the aHCA in the manner of an alcoholysis. The conversion of the aHCA is preferably carried out by alcoholysis with an alcohol preferably comprising 1-10 carbon atoms, more preferably 1 to 5 carbon atoms. Preferred alcohols are, inter alia, methanol, ethanol, propanol, butanol, in particular n-butanol and 2-methyl-1-propanol, pentanol, hexanol, heptanol, 2-ethylhexanol, octanol, nonanol and decanol. The alcohol used is more preferably methanol and/or ethanol, methanol being very particularly advantageous. It is also possible in principle to use precursors of an alcohol. Alkyl formates may be used, for example. Methyl formate or a mixture of methanol and carbon monoxide are particularly useful.

For the purposes of the invention, the reaction between aHCA and alcohol is carried out in a pressure reactor in the liquid phase. This should in principle be understood to mean a reaction space that allows a positive pressure to be maintained during the reaction. For the purposes of the invention, the pressure reactor is preferably configured as a tubular reactor. Tubular reactors are known to those skilled in the art and are described for example in Cresswell, D., Gough, A. and Milne, G., 2000, Tubular Reactors, Ullmann's Encyclopedia of Industrial Chemistry.

Positive pressure in this context means a pressure greater than atmospheric pressure, i.e. in particular greater than 1 bar. For the purposes of the invention the pressure can be in a range from greater than 1 bar to less than or equal to 100 bar, preferably 10-90 bar, more preferably 20-70 bar and yet more preferably 30-65 bar. Thus, the pressure is greater than atmospheric pressure or greater than 1 bar both during the inventive reaction/alcoholysis of the alpha-hydroxycarboxamide and during the separating-off/removal of the ammonia from the product mixture. In particular, this means that the ammonia formed in the reaction is also distilled out of the mixture under a pressure of greater than 1 bar, the use of assistants such as stripping gas for the distillative removal of the ammonia being entirely dispensed with. The best separation results for ammonia and methanol without stripping medium are obtained when the pressure is less than the reactor pressure but greater than 1 bar.

For the purposes of the invention the product mixture is depleted not only in ammonia but also in unreacted alcohol. Specifically in the case that methanol is used for the alcoholysis, a product mixture which comprises, inter alia, the components ammonia and methanol which are in principle very difficult to separate from one another results. In the simplest case, the product mixture is depleted in ammonia and alcohol by directly removing said two components as a substance mixture from the product mixture. The two substances are then subjected to a downstream separating operation, for example a rectification. Alternatively, it is also possible for the purposes of the invention to separate the two components alcohol (methanol) and ammonia from the product mixture in one procedure, and simultaneously also to separate the two constituents ammonia and alcohol (methanol) from one another.

It is, however, also possible in an alternative process variant to initially draw off only the ammonia as described in EP 945423 for example. There, the alcoholysis is effected in a stirred tank (CSTR) equipped with a column, the ammonia formed being continually removed from the reaction mixture via the column, at slightly elevated pressure. A consistently low concentration of ammonia advantageous for the reaction equilibrium can thus be established.

In a preferred process variant of the invention, it may be of particular interest that the reaction step and the removal of the ammonia/alcohol from the product mixture are spatially separated and carried out in different plants. To this end, for example, one or more pressure reactors may be provided and connected with a pressure distillation column. These reactors are one or more reactors disposed outside the column in a separate area.

In a further preferred process variant according to the invention, the reaction in the pressure reactor is repeated one or more times with the ammonia and alcohol depleted product mixture toward the bottom of the separation column (pressure distillation column), the reaction step being moved to a plurality of pressure reactors connected in series.

It is therefore of particular interest that the mixture depleted in ammonia and is withdrawn from a plate above the bottom of the distillation column, compressed to a pressure greater than the pressure in the distillation column and subsequently fed into a second pressure reactor, from where, after another reaction under the action of elevated pressure and temperature to obtain a twice-reacted product mixture, it is in turn decompressed to a pressure less than the pressure in the second pressure reactor and greater than 1 bar and subsequently fed back into the distillation column below the plate from which the feeding into the second pressure reactor was effected but above the bottom of the distillation column, where ammonia and alcohol are again distilled off overhead to obtain a mixture twice depleted in ammonia and alcohol.

This process step can be repeated as desired, with three to four repetitions, for example, being particularly favourable. In this regard, preference is given to a process which is characterized in that the reaction in the pressure reactor, the decompression of the reacted mixture, the feeding into the first distillation column, the depletion in ammonia and alcohol in the first distillation column, withdrawal of the depleted mixture, compression and feeding of the depleted mixture into a further pressure reactor are repeated more than once, wherein at the bottom of the pressure distillation column a product mixture which has been depleted n times in ammonia and alcohol according to the number n of pressure reactors connected in series is obtained. n may be a positive integer greater than zero. n is preferably in the range of 2 to 10.

Various temperature ranges in the column and the reactor have proven themselves to be particularly advantageous for the stated process variant.

Thus the pressure distillation column generally has a temperature in the range of about 60° C. to 220° C., preferably 80° C.-190° C., with 90° C.-180° C. being very particularly preferred. The exact temperature is typically established by the boiling system as a function of the prevailing pressure conditions.

The temperature in the reactor is preferably in the range of about 120-240° C. It is very particularly advantageous to keep the temperature constant from reactor to reactor at the start of the reaction when the catalyst is fresh. After prolonged reaction times it is advantageous to raise the temperature in the front reactors, for example in steps of 1-15° C. This positively influences the selectivity of the reaction and also the operating lifetime of the catalysts and keeps the degree of conversion of the reaction consistently high.

A further measure for increasing the selectivity can consist in reducing the amount of catalyst from reactor to reactor. A decreasing amount of catalyst with an increasing total degree of conversion also results in improved selectivity.

In a particular variant of the process according to the invention, it is favourable to withdraw the product mixture to be withdrawn from the pressure distillation column at particular points of the column. The distance between the withdrawal point and the bottom of the column is used for guidance as a relative indication of location. In the context of the invention it is particularly advantageous to feed the decompressed product mixture of step b) into a pressure reactor more closely adjacent to the bottom of the distillation, relative to the feed point of the feeding of the previous step b), after each new reaction.

The reaction temperature may likewise vary over a wide range, the reaction rate generally increasing with increasing temperature. The upper temperature limit generally arises from the boiling point of the alcohol used as a function of the established pressure. The reaction temperature is preferably in the range of 40-300° C., more preferably 120-240° C.

In the context of the invention it has been found that the outlined procedure can tolerate a broad spectrum of quantity ratios of the reactants. The alcoholysis can thus be carried out with a relatively large excess or deficiency of alcohol based on the aHCA. Process variants in which the reactants are reacted with a starting molar ratio of alcohol to aHCA in the range of 1:3 to 20:1 are particularly preferred. The ratio 1:2 to 15:1 is very particularly advantageous and 1:1 to 10:1 is yet more advantageous.

Further, process variants characterized in that the aHCA used is alpha-hydroxyisobutyramide and the alcohol used is methanol are preferred.

In the process according to the invention the alcoholysis of the aHCA takes place in the presence of a heterogeneous catalyst.

Preferred process variants are those in which the catalyst is an insoluble metal oxide comprising at least one element selected from the group consisting of Sc, V, La, Ti, Zr, Y, Hf, V, Nb, Ta, Cr, Mo, W, Tc, Re, Fe, Co, Ni, Cu, Al, Si, Sn, and Pb.

Catalysts based on ZrO₂ and Al₂O₃ are particularly preferred, with the use of ZrO₂ catalysts doped with lanthanum oxide, silicon oxide or yttrium oxide being very particularly preferred. The latter are commercially available for example as zirconium oxide catalyst SZ 61157 from Saint-Gobain Norpro. The yttrium inserted in the zirconium oxide crystal lattice effects a stabilization of the tetragonal phase of the zirconium oxide, which is otherwise only stable above 1200° C., at room temperature. These are used industrially as oxygen conductors for solid oxide fuel cells or in oxygen measuring devices (λ-sensor). A composition having 8 mol % of Y₂O₃ is typical here. In the process according to the invention, lanthanum oxide, silicon oxide or yttrium oxide contents of 0.05-20 mol %, preferably of 0.5-15 mol %, more preferably 1-10 mol % and yet more preferably of 2-5 mol % based on ZrO₂ are used. Mixtures of the catalysts mentioned may also be used.

When using Al₂O₃, doping with BaO has proved successful. Good results are achieved with 0.01-1.2 mol % of BaO based on Al₂O₃. Particular preference is given to 0.05-1.0 mol % and 0.1-0.8 mol % is very particularly preferred.

It was found, surprisingly, that these catalysts have a high tolerance to the presence of water. In the alcoholysis reaction, the water content in the reactant feed may thus be 0.1-20 wt %. Preference is given to 0.5-10 wt %, 0.8-3 wt % are particularly preferred.

The heterogeneous catalysts according to the invention are configured as a fixed bed within the abovementioned pressure reactor. Embodiments of catalytic fixed bed reactors are known to those skilled in the art and are described for example in Eigenberger, G. and Ruppel, W., 2012, Catalytic Fixed-Bed Reactors, Ullmann's Encyclopedia of Industrial Chemistry.

In the context of the present invention the use of the catalyst in at least one, preferably in more than one fixed bed(s) in series has proved advantageous, the latter then each being provided with an intermediate step—for example introduction into the above mentioned pressure distillation column—for the ammonia depletion.

In a further variant of the process according to the invention, the temperature in the catalyst fixed bed is adjusted as a function of conversion in that the reaction temperature is increased as a function of the decrease in the degree of conversion. Depending on the catalyst used, given a decline in conversion from 48% to 37% for example, the original value of the conversion can be re-established with a temperature elevation of 5° C.

In the particularly preferred variant of the process according to the invention, the methanolysis of alpha-hydroxyisobutyramide (HIBAm) to form methyl alpha-hydroxyisobutyrate (MHIB), it was found, surprisingly, that under the reaction conditions mentioned a high tolerance further exists not only for the presence of water but also for the impurities present in the reaction feed from the previous stage—hydrolysis of acetocyanohydrin (ACH) to form alpha-hydroxyisobutyramide (HIBAm) via manganese oxide catalysis—such as alpha-hydroxyisobutyric acid (HIBAc), alpha-aminoisobutyramide (A-HIBAm), formamide (FA) or tetramethyloxazolidinone (TMO). The sum of these impurities may thus amount to a maximum of 10 wt % based on HIBAm, preferably a maximum of 5 wt % and more preferably a maximum of 3 wt % in the reaction feed without significant efficiency losses in the alcoholysis reaction.

In the aformentioned production of MHIB by the process according to the invention, numerous by-products occur in the product mixture from step c). In particular the ammonium salt of HIBAc (Am-HIBAc), alpha-hydroxyisobutyric acid methyl amide (HIBMAm), methyl alpha-methoxyisobutyrate (MMIB) or alpha-methoxyisobutyric acid methyl amide (MIBMAm) may be mentioned here. It was found, surprisingly, that these by-products may also be fed into the feed of the methanolysis step a) together with the unreacted alpha-hydroxyisobutyramide (HIBAm) and the unreacted excess methanol isolated elsewhere, without the selectivity or conversion of the catalyst declining. It is immaterial in which of the possible repetition stages of reaction step b) this feeding is carried out, and distribution over two or more of the repetition stages is alternatively possible. The recycling is preferably effected into the first stage of step b). The sum of the fractions of all by-products present in the product mixture from step c) should be no more than 85 wt %, preferably no more than 65 wt % and more preferably 50 wt % of the total feed in step a).

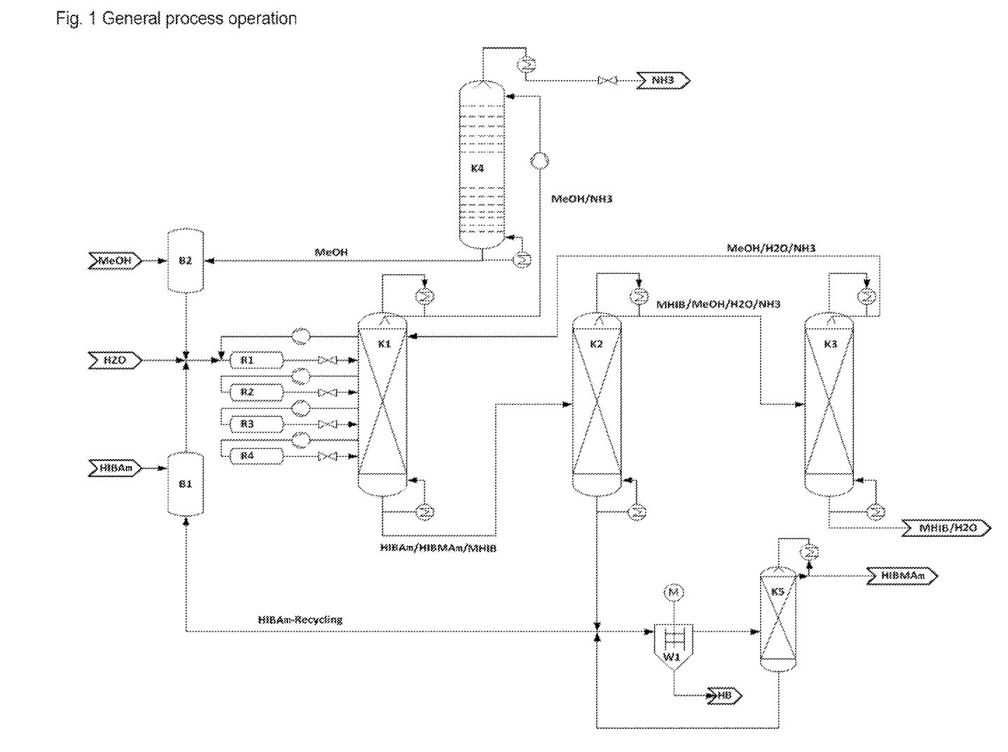

In an exemplary general process operation of one of the process variants according to the invention (FIG. 1), HIBAm is metered into a reservoir vessel (B1) where it is merged with two recycle streams (from K2 and K5). The mixture obtained is metered to the reactor R1 (Loop 1) from this reservoir vessel. Depending on the water content of the mixture fed into the reactor, additional water may be fed into Loop 1. Methanol (MeOH) required for the reaction is provided firstly as recycle-MeOH via the runback of K4, and secondly as fresh MeOH via metering from a reservoir vessel (B2). All loops are operated at 200° C. The weight fractions of the catalyst distribution over the individual loops are 2.7:2.0:1.3:1.0.

The partial reaction of HIBAm and MeOH to form MHIB and NH₃ takes place in R1. The output stream is passed into column K1 whereupon NH₃ partially evaporates. The reaction is repeated in three further reactors (R2 to R4), wherein the MHIB content increases in a downwards direction. The space velocity over the catalyst likewise increases in a downward direction due to decreasing amounts of catalyst. Space-time yield and selectivity are thereby kept constant. The output stream of the last reactor R4 is largely freed of MeOH and NH₃ in the stripping section of K1. In the rectification section of K1, NH₃ from all reaction steps concentrates to 8-10 wt % in MeOH depending on pressure. A distillate of this quality is passed from K1 into the column K4, at the top of which gaseous NH₃ accumulates and at the bottom of which NH₃-free MeOH is withdrawn and passed into the MeOH reservoir (B2). A mixture of HIBAm, MHIB, and by-products accumulates in the bottom of K1 and is passed into column K2. There, MHIB with residual water, MeOH and NH₃ components is distilled overhead and accumulates in about 85-90 wt % purity. The distillate is passed into a further column K3, in which MeOH and NH₃ are driven off overhead and from there recycled into K1. In the bottom of K3, MHIB accumulates as pure product, with water, for further processing. In the bottom of K2, unreacted HIBAm is withdrawn in concentrated form together with the by-products mentioned. The majority (about 95-97%) of this stream is recycled directly into the HIBAm reservoir (B1). The remainder is passed over the thin-film evaporator W1, in the bottom of which high boiling by-products and inorganic trace components (HB) are separated off. The vapours of W1 are passed into the column K5, at the top of which HIBMAm is discharged from the process in concentrated form. A HIBAm-rich stream purified of high boiling by-products and partially purified of HIBMAm accumulates in the bottom and is metered into the HIBAm reservoir (B1).

The following examples are intended to illustrate the invention without limiting it in any way.

### EXAMPLES 1-9

### Catalyst Variants

The experiments of Examples 1-9 were carried out in an electrically heated fixed-bed stainless steel reactor (di=10 mm). In each case, 16 g of the catalysts described in Table 1 were initially charged as the catalyst material, typically in the commercially available form as extrudates. Inert glass wool was installed below and above the bed as an inlet and an outlet zone.

As feed, a mixture of MeOH/HIBAm (98.5% pure)=7:1 molar was passed from below over the bed in which a temperature of 220° C. had been established, with a feed rate of 2 ml/min. A pressure of 60 bar a was established via a pressure retention valve.

For sample collection, a substream was withdrawn from the reactor output stream and cooled with dry ice. The analysis of the samples was carried out by means of gas chromatography. See appendix for apparatus and method.

TABLE 1

| | | | X_HIBAm | | |
|---|---|---|---|---|---|
| Example | Catalyst | Producer | in % | Y_MHIB in % | S_MHIB* in % |
| E1 | ZrO₂ + Y₂O₃ | Saint-Gobain | 38.10 | 34.90 | 91.60 |
| | (SZ61157) | | | | |
| E2 | ZrO₂ + Y₂O₃ | Evonik | 36.90 | 34.50 | 93.50 |
| E3 | ZrO₂ + SiO₂ | Saint-Gobain | 38.40 | 33.20 | 86.50 |
| | (SZ61152) | | | | |
| E4 | ZrO₂ + La₂O₃ | Saint-Gobain | 35.80 | 32.90 | 91.90 |
| | (SZ61156) | | | | |
| E5 | ZrO₂ | Evonik | 27.60 | 25.20 | 91.30 |
| E6 | Al₂O₃ + BaO | Evonik | 22.20 | 20.50 | 92.30 |
| | 0.10 wt % | | | | |
| E7 | Al₂O₃ | Evonik | 21.60 | 19.70 | 91.20 |
| E8 | Al₂O₃ Puralox | Sasol | 21.60 | 17.80 | 82.40 |
| | SCCa 150_200 | | | | |
| E9 | Al₂O₃ + BaO | Evonik | 17.50 | 16.70 | 95.40 |
| | 0.50 wt % | | | | |

The best yields of MHIB (Y-MHIB) are achieved with zirconium oxide catalysts. The best performance is achieved with yttrium oxide, lanthanum oxide and/or silicon oxide dopants. These are followed by the aluminium oxides which show selectivity advantages (S-MHIB) when doped with small amounts of barium oxide.

(* Not considered in this selectivity are Am-HIBAc, HIBMAm, MIBMAm and MMIB, which contribute to the total yield in the manner of a material of value in a circulation process.)

### Comparative Examples 1-8

### Catalyst Variants

The comparative experiments for the Examples 1-8 were carried out according to the inventive Examples 1-9 with different catalysts.

TABLE 2

| Comp. | | | MeOH/ | X_HIBAm | Y_MHIB | S_MHIB* |
|---|---|---|---|---|---|---|
| Example | Catalyst | Producer | HIBAm | in % | in % | in % |
| CE1 | TiO₂ | Evonik | 10 | 43.20 | 25.00 | 57.90 |
| CE3 | Al₂O₃ + BaO | Evonik | 7 | 13.80 | 11.30 | 81.90 |
| | 1.50 wt % | | | | | |
| CE2 | ZnO | Evonik | 7 | 15.00 | 10.10 | 67.30 |
| CE4 | CeO₂ | Sigma- | 10 | 11.00 | 8.50 | 77.30 |
| | (211575) | Aldrich | | | | |
| CE5 | Sb₂O₃ | Sigma- | 7 | 8.50 | 8.00 | 94.10 |
| | (230898) | Aldrich | | | | |
| CE6 | Bi₂O₃ | Sigma- | 10 | 7.20 | 5.60 | 77.80 |
| | (223851) | Aldrich | | | | |
| CE7 | TiO₂ + Bi₂O₃ | Sigma- | 10 | 5.80 | 5.50 | 94.80 |
| | (403687) | Aldrich | | | | |
| CE8 | ZrO₂ + Bi₂O₃ | Sigma- | 10 | 5.20 | 4.30 | 82.70 |
| | (403660) | Aldrich | | | | |

The high conversion with metal salts such as CeO₂, Sb₂O₃ or Bi₂O₃, as described in the prior art in JP 08073406 and JP 06345692, cannot be verified here.

### EXAMPLE 10

### Influence of Water on Kinetics

A reactor with tapping points (length 2 m, interior diameter=23 mm, catalyst inventory 1 kg Zr₂O₃+Y₂O₃; Saint-Gobain) was operated with 0.3, 1.0 and 2 wt % of water. The molar ratio of MeOH:HIBAm was 7:1, temperature 200° C. and pressure 60 bar. The feed rate was converted for the tapping points so that the (modified) residence time τ_{mod} (mass of catalyst/feed rate) could be plotted. An increase in the degree of conversion with increasing water amount (X-HIBAm) can be measured:

TABLE 3

| T_{mod} (g min/ml) | 0.3 wt % water | 1.0 wt % water | 2.0 wt % water |
|---|---|---|---|
| 1.3 | 3.8% | 3.3% | 7.1% |
| 2.3 | 6.0% | 7.5% | 9.6% |
| 4.3 | 11.3% | 13.2% | 15.4% |
| 5.3 | 14.1% | 15.1% | 17.6% |
| 7.3 | 16.9% | 18.1% | 20.8% |
| 8.3 | 18.5% | 20.3% | 22.1% |
| 10.0 | 22.5% | 22.5% | 24.5% |
| 10.7 | 22.5% | 22.7% | 24.2% |
| 12.0 | 24.1% | 23.7% | 25.7% |
| 12.7 | 24.1% | 23.6% | 25.7% |

### EXAMPLES 11-12

### Utility of by-Products

The Examples 11-12 were run with (Zr₂O₃+Y₂O₃) catalyst according to Example 1 at T=200° C., p=60 bar and a feed rate of 2 ml/min.

TABLE 4

| | | MeOH | HIBAm | Am-HIBAc | MHIB | H₂O | NH₃ |
|---|---|---|---|---|---|---|---|
| Example | Component | [wt %] | [wt %] | [wt %] | [wt %] | [wt %] | [wt %] |
| 11 | Reactant | 70 | 0 | 29 | 0 | 0.25 | 0 |
| | feed | | | | | | |
| | Product | 66.5 | 15.3 | 1.7 | 10.2 | 4.8 | 1.5 |
| | mixture | | | | | | |
| 12 | Reactant | 68 | 31 | 0.20 | 0 | 0.25 | 0 |
| | feed | | | | | | |
| | Product | 64.7 | 22.7 | 0.24 | 9.7 | 0.38 | 1.5 |
| | mixture | | | | | | |

As is shown by the experiments, Am-HIBAc may also be used as feed for the methanolysis (step b)) in the process according to the invention. An equilibrium state similar to that of the standard mode of operation (Example 12) arises—a large proportion of Am-HIBAc is converted back to HIBAm—which is, however, shifted slightly due to the comparatively high water content. The yield of MHIB is of similar magnitude and is in the range of 25-30%.

Ammonium HIBAc (Am-HIBAc) analysed by means of HPLC. Ammonia determination by means of titration, water content determination according to Karl-Fischer.

### EXAMPLE 13

### HIBMAm to MHIB

Example 1 was repeated with a feed consisting of 9.5 wt % HIBMAm in MeOH which was passed over an Y₂O₃+ZrO₂ catalyst from Saint-Gobain Norpro with a feed rate of 2 ml/min at 220° C. Analysis carried out by GC (see appendix).

A degree of HIBMAm conversion of 19% was measured under the conditions indicated. The reaction proceeded selectively to form MHIB (S=100%). The fact that HIBMAm also reacts to form the target product (albeit more slowly than HIBAm) allows operation at a stationary accumulation level with high yield of the material of value.

### EXAMPLES 14-19

### TMO to HIBAm

Example 1 was repeated at 210° C. with an Y₂O₃+ZrO₂ catalyst from Saint-Gobain Norpro.

Different amounts of water and also a defined amount of TMO (0.04 wt %) were added to the feed.

TABLE 5

| | Water in the feed | TMO in the product | |
|---|---|---|---|
| Examples 14-19 | in wt % | in wt % | X_TMO % |
| E14 | 0.74 | 0.04 | 0 |
| E15 | 0.99 | 0.03 | 12 |
| E16 | 1.52 | 0.02 | 53 |
| E17 | 1.98 | 0.01 | 63 |
| E18 | 3.02 | 0.01 | 60 |
| E19 | 4.01 | 0.01 | 67 |

The results clearly show that TMO is (only) converted as of a certain minimum concentration of water in the feed. The fact that TMO reacts to completion in the presence of water allows operation at a stationary accumulation level with high yield of the material of value.

### APPENDIX

### GC Analysis

| Component | Article designation |
|---|---|
| GC | CP-3800 gas |
| | chromatograph |
| Injector | |
| Front channel | 1079 PTV |
| Rear channel | 1177 split/splitless |
| Column | |
| Front channel | DB WAXERT (Agilent) |
| | length 30 m |
| | ID 0.53 mm |
| | film 1.5 μm |
| | +pre-column |
| Rear channel | AT-WAX (Alltech) |
| | length 30 m |
| | ID 0.53 mm |
| | film 1.2 μm |
| Detector | |
| Front channel | TCD (thermal conductivity |
| | detector) |
| Rear channel | FID |
| | (flame ionization detector) |

The temperature program provides heating from 40-230° C. at 15 K/min.

### HPLC Analysis

| Component | Article designation |
|---|---|
| Pump | Pump: Dionex HPLC |
| | pump 680 |
| Column oven | Thermostated column |
| | compartment TCC-100 |
| UV detector | Ultimate 3000 variable |
| | wavelength detector |
| HPLC column | Zorbax SB-Aq Rapid |
| | Solution 4.6 × 150 mm; |
| | 3.5 micron |
| Eluent | Acetonitrile LiChrosolv |
| | from Merck |
| | Potassium |
| | dihydrogenphosphate |
| | solution (0.02 mol/l) |

Temperature 70° C.

1. Continuous process for the production of alpha-hydroxycarboxylic esters by alcoholysis of the corresponding alpha-hydroxycarboxamide, wherein
a) Reactant streams comprising an alpha-hydroxycarboxamide and an alcohol are fed into a pressure reactor containing a heterogeneous catalyst,
b) this reaction mixture is co-reacted in the pressure reactor at a pressure in the range of 1-100 bar in the liquid phase,
c) the product mixture resulting from step b) comprising alpha-hydroxycarboxylic ester and also unreacted alpha-hydroxycarboxamide is discharged from the pressure reactor,
d) the product mixture resulting from c) is depleted in alcohol and ammonia, and
e) the unreacted reactants and also by-products comprising ammonium salts of the corresponding alpha-hydroxycarboxylic acid, present in the product mixture from step d), are recycled to step a).

2. The process according to claim 1, wherein the reaction of the alpha-hydroxycarboxamide is effected with a heterogeneous catalyst based on ZrO₂ and/or Al₂O₃.

3. The process according to claim 2, wherein the ZrO₂ employed is doped with 0.1-20 mol % of Y₂O₃ or La₂O₃ or SiO₂.

4. The process according to claim 2, wherein the Al₂O₃ employed is doped with 0.01-1.2 mol % of BaO.

5. The process of claim 1, wherein the molar ratio of alcohol to alpha-hydroxycarboxamide is 1:3 to 20:1.

6. The process of claim 1, wherein the water content in the reactant feed is 0.1-20 wt %.

7. The process of claim 1, wherein the by-products in step e) comprise HIBMAm and/or TMO.

8. The process of claim 1, wherein the reactant streams are co-reacted in a pressure reactor at a pressure in the range of 1-100 bar.

9. The process of claim 1, wherein the ammonia formed is distilled off at a pressure which is continually kept at greater than 1 bar and less than the pressure in the pressure reactor, without the aid of additional stripping media.

10. The process of claim 1, wherein the reaction temperature is increased as a function of the decrease in the degree of conversion.

11. The process of claim 1, wherein when pressure reactors are connected in series, the amount of catalyst decreases from reactor to reactor.
